(19) United States
(12) Patent Application Publication
(10) Pub. No.: US 2005/0273038 A1
(43) Pub. Date: Dec. 8, 2005

(54) Tampon applicator providing low placement

(75) Inventors: Thomas Ward Osborn III, Clifton, OH (US); Diana Lynne Gann, Lebanon, OH (US); Charles John Berg JR., Wyoming, OH (US); Jacqueline Ann Daniels, Fairfield, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/860,921

(22) Filed: Jun. 4, 2004

(51) Int. Cl. A61F 13/20

(52) U.S. Cl. 604/15

(57) ABSTRACT

A tampon applicator for positioning a tampon inside a vaginal cavity. The tampon applicator includes an outer member, an inner member which is slidable within the outer member, and a tampon which is housed within the tampon applicator. The inner member has an effective length of less than about 58 mm.

### FIELD OF INVENTION

This invention relates to a tampon applicator, particularly, to a tampon applicator providing low placement of a tampon within the vaginal cavity.

### BACKGROUND OF THE INVENTION

Tampon applicators have been used for inserting a tampon into a vaginal cavity to prevent leakage of menstrual fluids or other fluids. In the field of tampon applicators, “push” type tampon applicators are well known. These applicators generally comprise, in a simple form, a pair of coaxial hollow cylinders or tubes. The larger outer tube serves as a vaginal insertion tube and houses a tampon. The smaller inner tube serves as a plunger which ejects the tampon from the outer tube.

It has been recognized that tampon applicators of the type described above have certain deficiencies which can be readily apparent to many users of such devices. In general, the position of the tampon within the vaginal cavity can directly impact the tampon's efficiency of reducing and/or eliminating leakage. For example, conventional “push” type applicators can place a tampon too high in the vaginal cavity. The higher the tampon is placed, the greater the chance of leakage due to the tampon being above the major path of fluid flow. In other words, menstrual fluids can pass by the tampon without being absorbed.

In addition, if the position of the tampon is too high in the vaginal cavity, the tampon can extend into portions of the vaginal cavity, where, for reasons of the size and mechanical properties of the tampon, the tampon can be negatively affected by the vaginal and surrounding organs, further reducing the tampon coverage of menses flow in the vaginal cavity. Moreover, current tampon applicators are designed to “push” the tampon out of the outer tube substantially higher than the tip of the outer tube. This often causes the tampon to be deflected by the cervix/anterior fornix, resulting in an off-centered position of the tampon, and, thus, result in possible leakage from the vaginal cavity.

Alternatively, tampon applicators can place a tampon too low in the vaginal cavity. When the tampon is placed too low in the vaginal cavity, the too low position of the tampon can cause bodily discomfort to the wearer because of the pressure exerted from the sphincter muscles against the tampon. Also, if the tampon is placed too low, accidental expulsion of the tampon is a highly undesired risk.

To solve the above problem of positioning the tampon, it would be beneficial to provide a tampon applicator which properly places the tampon at a desired position in the vaginal cavity.

### SUMMARY OF THE INVENTION

The present invention encompasses a tampon applicator for positioning a tampon inside a vaginal cavity. The applicator includes an outer member and an inner member slidable within the outer member. The inner member has an effective length of less than about 58 mm. The tampon is housed within the tampon applicator.

All documents cited are, in relevant part, incorporated herein by reference; the citation of any document is not to be construed as an admission that it is prior art with respect to the present invention.

### BRIEF DESCRIPTION OF THE DRAWINGS

FIG. 1 is a perspective view along a longitudinal axis of a tampon applicator of the present invention.

FIG. 2 is an exploded view of the tampon applicator along the longitudinal axis illustrative of the present invention before the assembly of the tampon applicator.

FIG. 3a is a perspective view of a tampon applicator of the present invention.

FIG. 3b is a cross-sectional view along line 3b-3b of the tampon applicator of FIG. 3a.

FIG. 4 is a perspective view of the tampon applicator of the present invention fully engaged.

FIG. 5 is a perspective view of an alternative embodiment of the tampon applicator of the present invention fully engaged.

FIG. 6a is a perspective view of an alternative embodiment of the tampon applicator of the present invention.

FIG. 6b is a perspective view of an alternative embodiment of the tampon applicator of the present invention fully engaged.

FIG. 6c is a perspective view of an alternative embodiment of the tampon applicator of the present invention fully engaged.

FIG. 7a is a perspective view of an alternative embodiment of the tampon applicator of the present invention.

FIG. 7b is a perspective view of an alternative embodiment of the tampon applicator of the present invention fully engaged.

FIG. 7c is a perspective view of an alternative embodiment of the tampon applicator of the present invention fully engaged.

### DETAILED DESCRIPTION OF THE INVENTION

Section A will provide terms which will assist the reader in best understanding the features of the invention but not to introduce limitations in the terms inconsistent with the context in which they are used in this specification. These definitions are not intended to be limiting. Section B will discuss the tampon applicator of the present invention. Section C will discuss the tampon applicator materials.

A. Terms

The term “fully engaged,” as used herein, occurs when the second end of the inner member is flush with the second end of the outer member or after 1250 grams of force has been applied to the end of the inner member.

The term “effective distance,” as used herein, is the distance that the trailing end of the tampon travels after the tampon pushing member contacts the tampon.

The term “effective length,” as used herein, is the end of the tampon to the second end of the outer member of the tampon applicator following full applicator engagement.

The term “stop,” as used herein, is meant when a member is used to prevent respective second ends of inner member and outer member from becoming flush with one another or when 1250 grams of force is applied to the inner member. One skilled in the art could imagine that the inner member or the outer member could have projections, nibs, ribs, nubs, etc. that can function to stop the inner member from being inserted into the outer member.

The term “exposed portion of the tampon,” as used herein, is meant the portion of the tampon that is revealed from the tampon applicator as measured from the leading end of the tampon to the insertion end of the outer member along the longitudinal axis when the applicator is fully engaged.

As used herein, the term “length of the outer member” refers to the length of the outer member measured along the longitudinal axis from the surface of the outer member which is closest to the body during insertion to the surface of the outer member which is furthest away from the body during insertion when the applicator is fully engaged.

As used herein, the term “tampon” refers to any type of absorbent structure that can be inserted into the vaginal cavity or other body cavities for the absorption of fluid therefrom or for the delivery of active materials, such as medicaments or moisture. A tampon can be straight or non-linear in shape, such as curved along the longitudinal axis.

Generally, there are two types of tampons. The first type of tampon is a self-sustaining tampon. Tampons are generally “self-sustaining” in that they will tend to retain their general shape and size before use. A typical self-sustaining tampon is 35-60 millimeters long, the length measured from the top of the tampon to the base of the tampon along a longitudinal axis. The measurement to the base of the tampon does not include any overwrap, secondary absorbent member, or withdrawal cord which extends beyond the tampon's main absorbent material. A typical self-sustaining tampon is 5-20 millimeters wide corresponding to the largest cylindrical cross section. The width can vary along the length of the self-sustaining tampon.

The second type of tampon is an easily “deformable, fluid-permeable bag tampon”. The deformable, fluid-perneable bag tampon consists of absorbent pieces such as chips, spheres, or fibers such that the fluid permeable bag tampon is readily deformable with a force of less than about 3 psi.

As used herein, the terms “vaginal cavity” and “within the vagina” refer to the internal genitalia of the human female in the pudendal region of the body.

As used herein, the term “hymen ring” refers to the demarcation between the vaginal cavity and external genitalia and is identified by the position of the hymen or residual tissue of the hymen.

As used herein, the term “low placement” refers to a position of the tampon inside the vaginal cavity, wherein the trailing end of the tampon is positioned at least about 5 millimeters above the hymen ring and below the cervix. The base of the tampon does not include any overwrap, secondary absorbent member, or withdrawal cord which extends beyond the tampon's main absorbent material.

The terms “expelled” and “expulsion,” as used herein, are meant the position of the tampon after the exposed portion of the tampon is fully or partially forced out of the tampon applicator.

The term “joined” or “attached” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element; i.e., one element is essentially part of the other element.

B. Tampon Applicator of the Present Invention

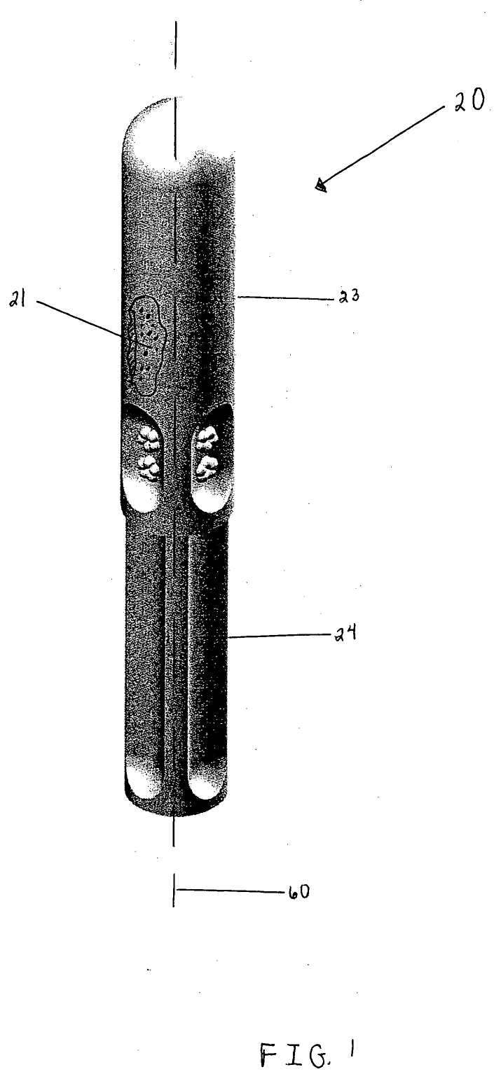

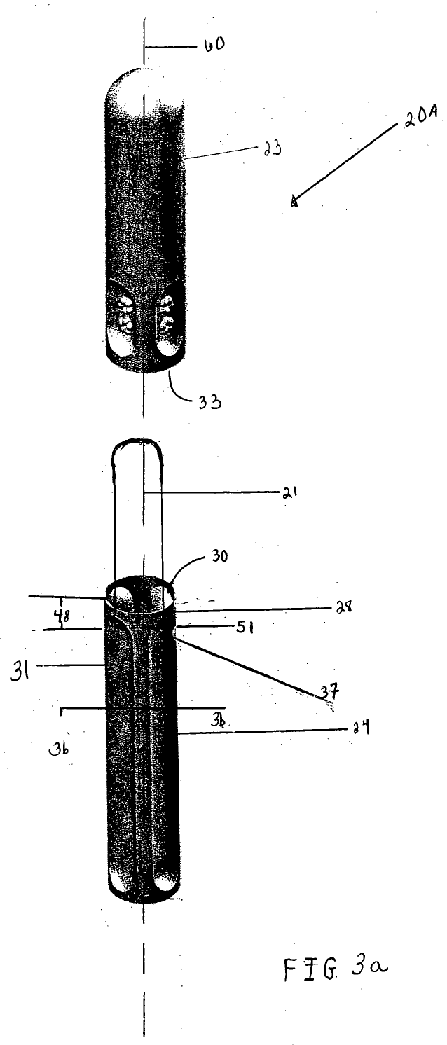

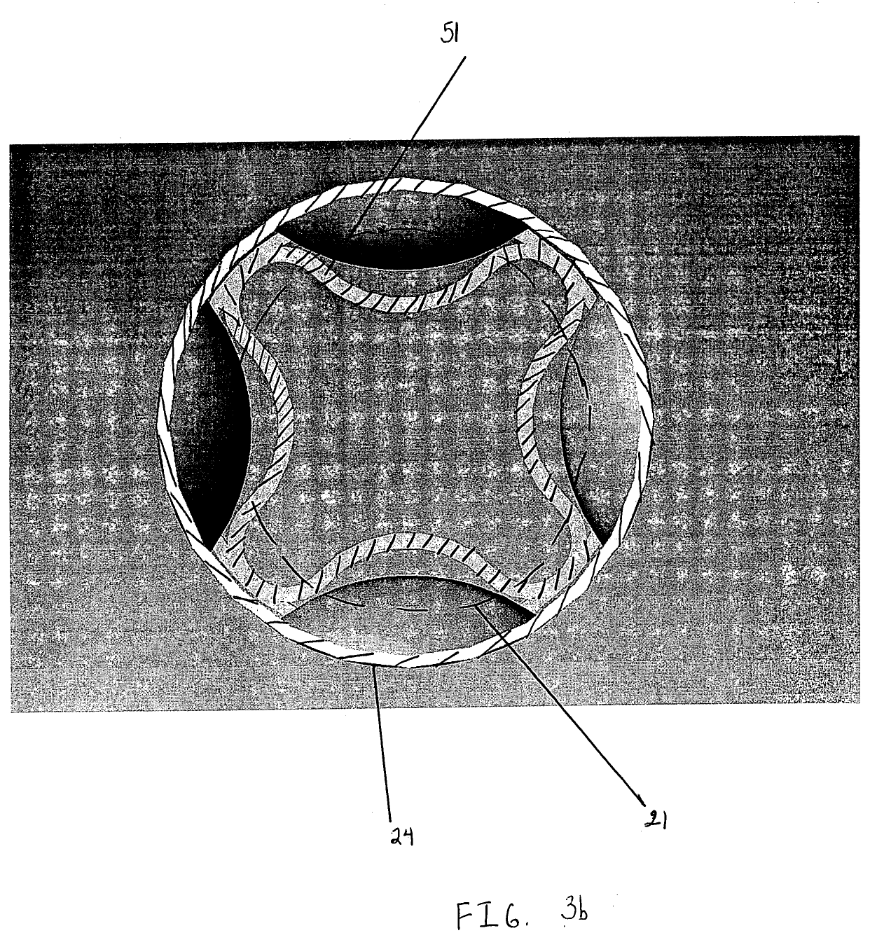

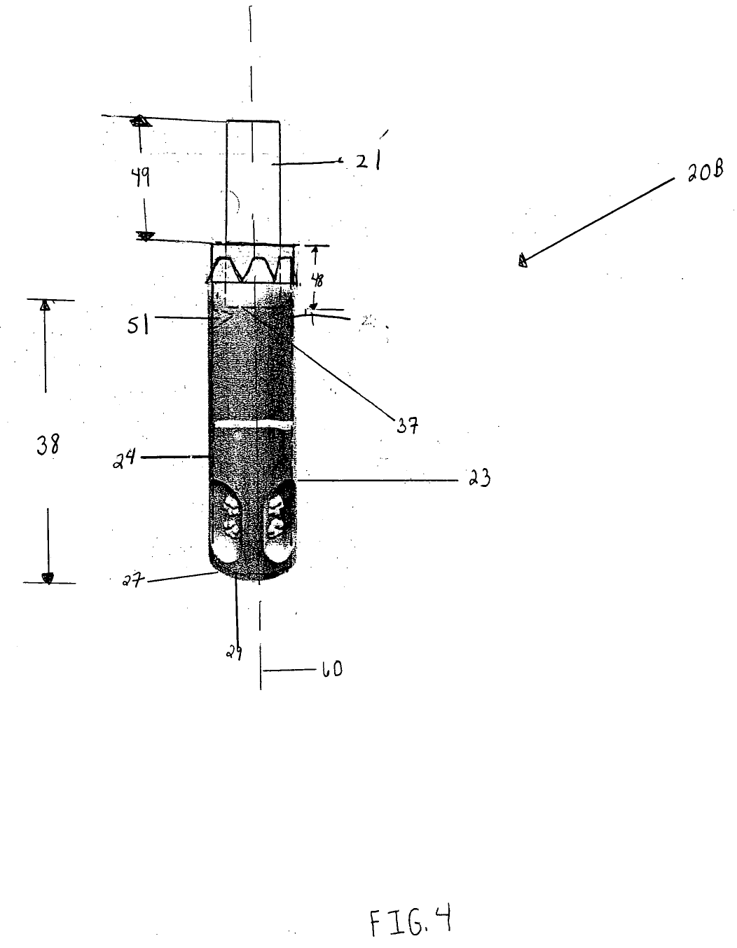

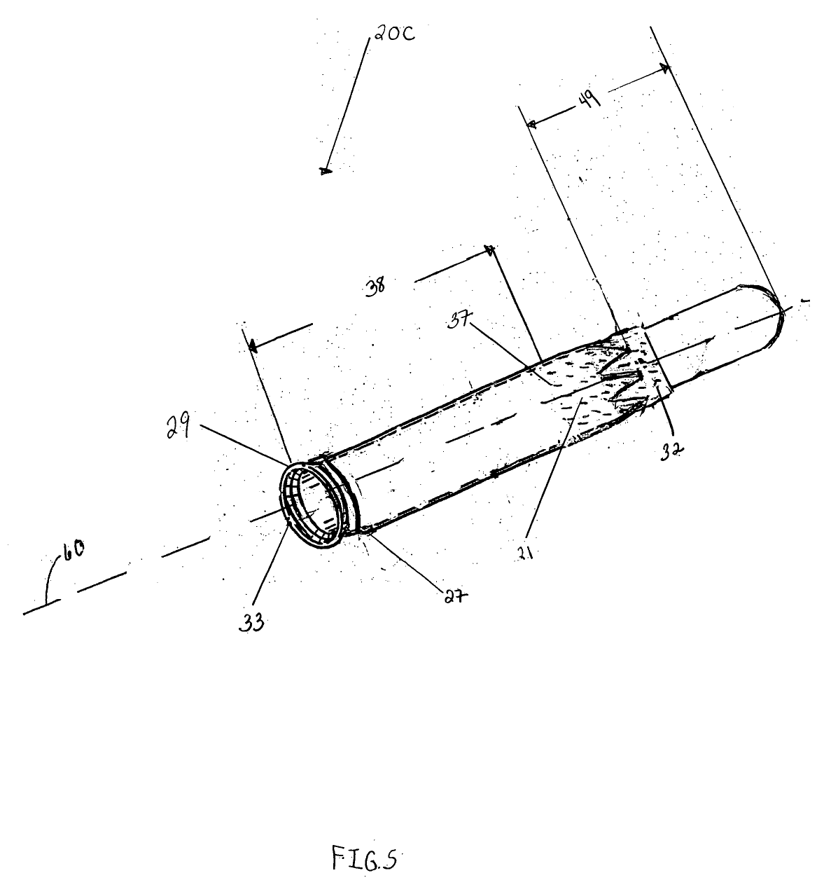

Referring to FIG. 1, a tampon applicator 20 is shown before expulsion of the exposed portion 49 (FIG. 4, FIG. 5) of tampon 21 along a longitudinal axis 60. The tampon applicator 20 is designed to position tampon 21 to achieve low placement in the vaginal cavity of a wearer. Generally, the tampon applicator 20 includes an outer member 23, an inner member 24, and a tampon pushing member 51 (see FIG. 3a).

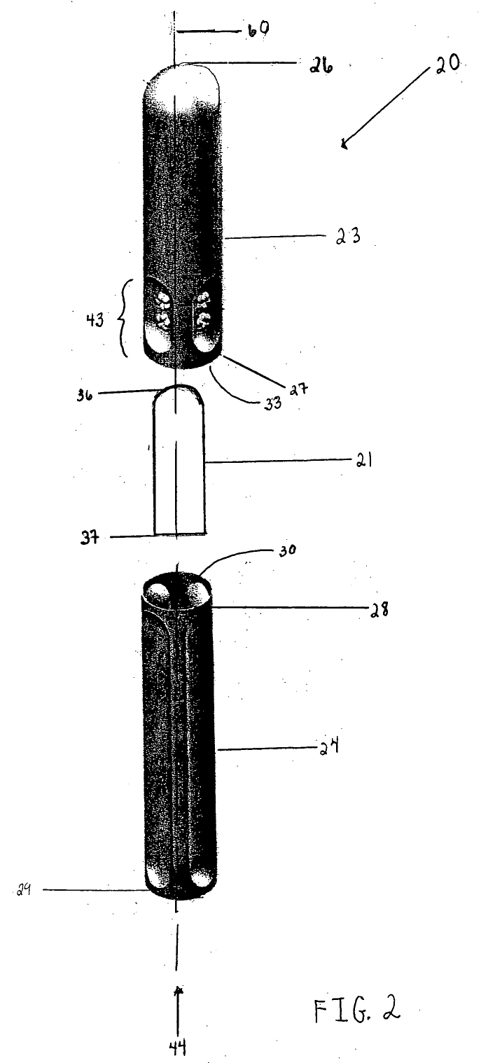

FIG. 2, is an exploded view of FIG. 1 of the tampon applicator 20 along the longitudinal axis 60. The inner member 24 having an effective length of less than 58 mm provides low placement of the tampon 21 within the vaginal cavity. In one non-limiting example, the effective length of the inner member is less than about 45 mm. In another non-limiting example, the effective length of the inner member is less than about 35 mm. In yet another non-limiting example, the effective length of the inner member is less than about 25 mm. Specifically, the effective length determines the placement depth of the trailing end 37 of the tampon 21 in the vaginal cavity. The trailing end 37 does not include any overwrap, secondary absorbent member, or withdrawal cord which extends beyond the tampon's main absorbent material. The outer member 23, the tampon 21, and the inner member 24 are shown. The outer member 23 can be used to insert the tampon 21 into the vaginal cavity. The outer member 23 comprises a hollow interior 33, an insertion end 26 dimensioned for insertion into the body cavity, and a second end 27 opposed to the insertion end 26. In addition, the outer member 23 can contain a grip region 43 located adjacent to the second end 27.

The tampon 21 has a leading end 36 and a trailing end 37. The leading end 36 of the tampon 21 is the end of the tampon 21 which is first inserted into the body. The trailing end 37 of the tampon 21 is opposed to the leading end 36 of the tampon 21. The tampon applicator 20 can be used with any type of tampon 21. For example, the tampon 21 could be a self-sustaining tampon or a deformable, fluid-permeable bag tampon.

The inner member 24 assists in the ejection of the exposed portion 49 (FIG. 4, FIG. 5) of the tampon 21 from the outer member 23 when a force 44 is applied along the longitudinal axis 60. The inner member 24 is dimensioned to slidably move within the outer member 23, with minimal clearance therebetween. The inner member 24 has a hollow interior 30. Alternatively, the inner member 24 can be solid or partially solid. The inner member 24 has a first end 28 and a second end 29 opposed to the first end 28.

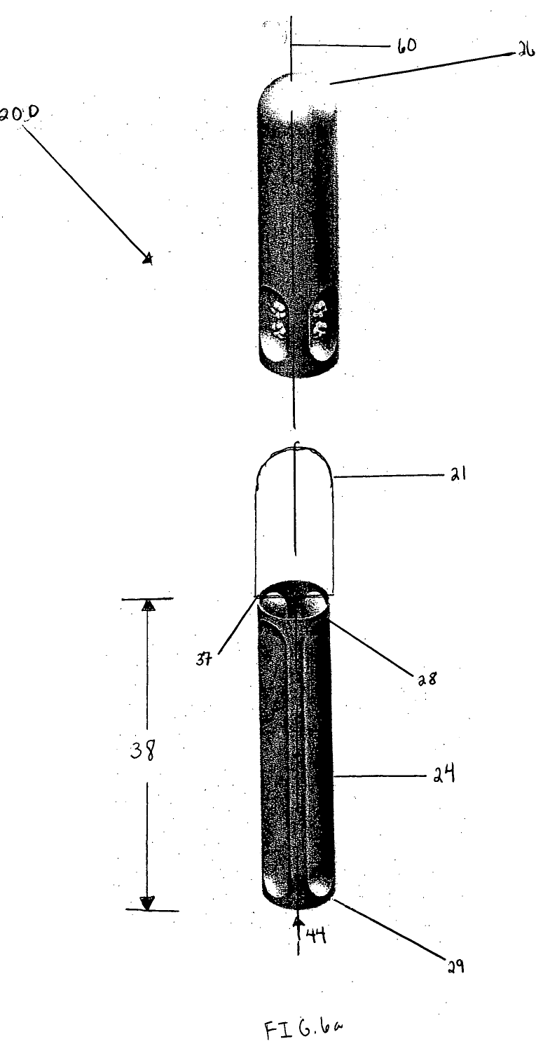

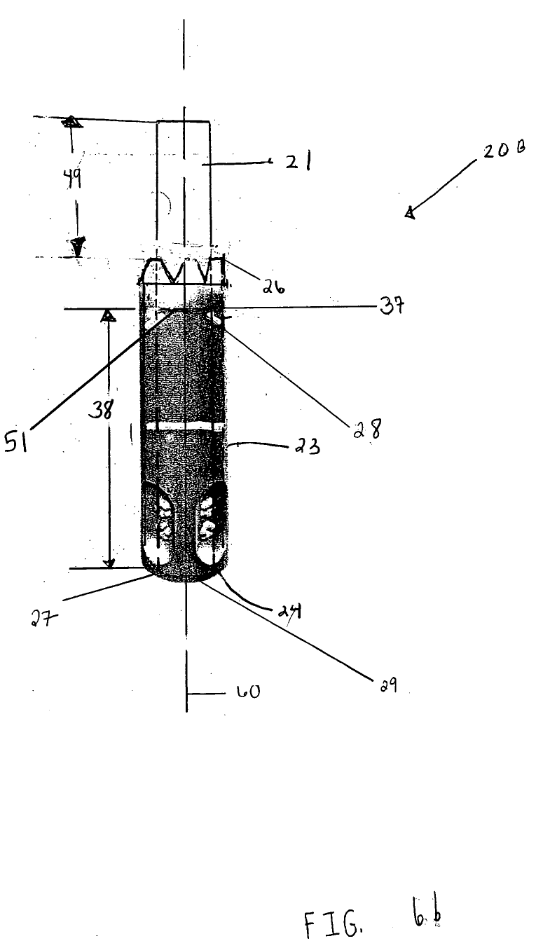

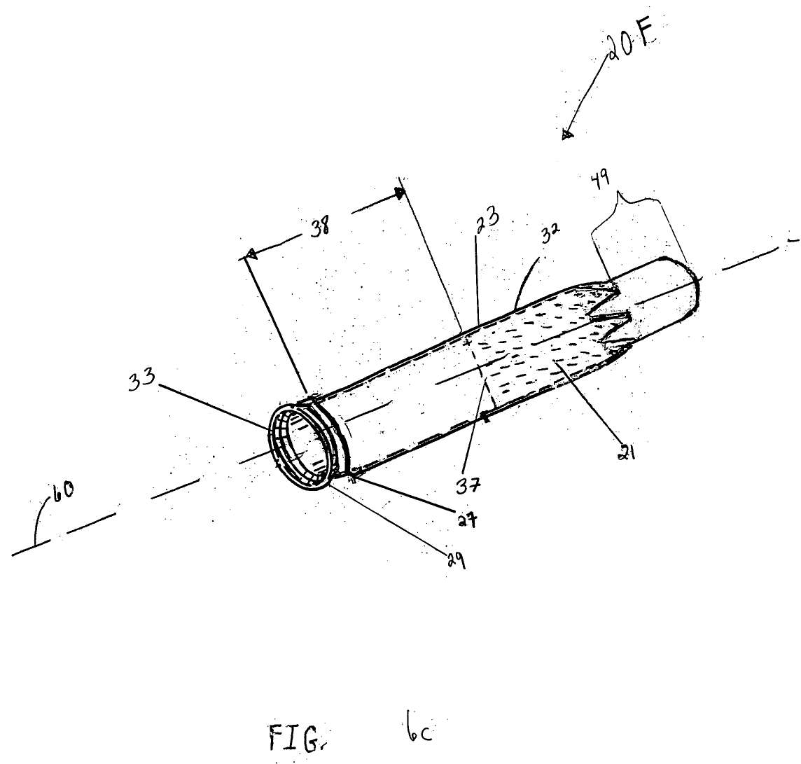

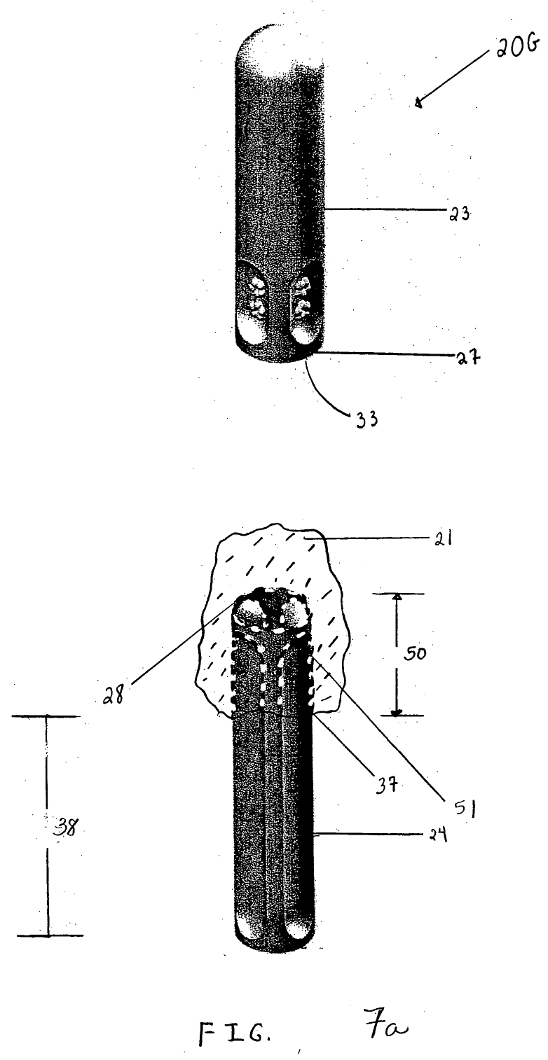

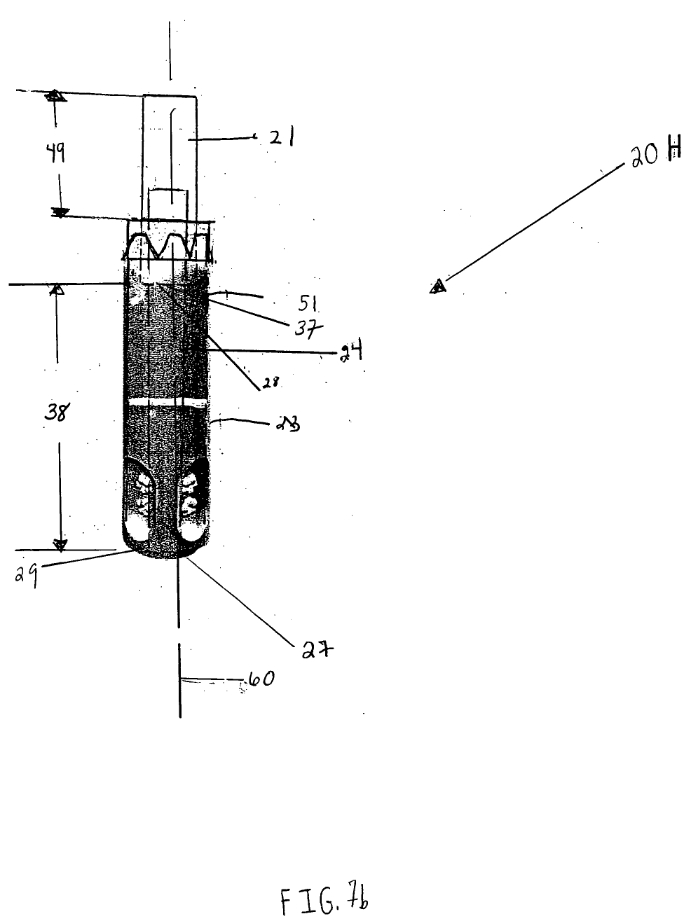

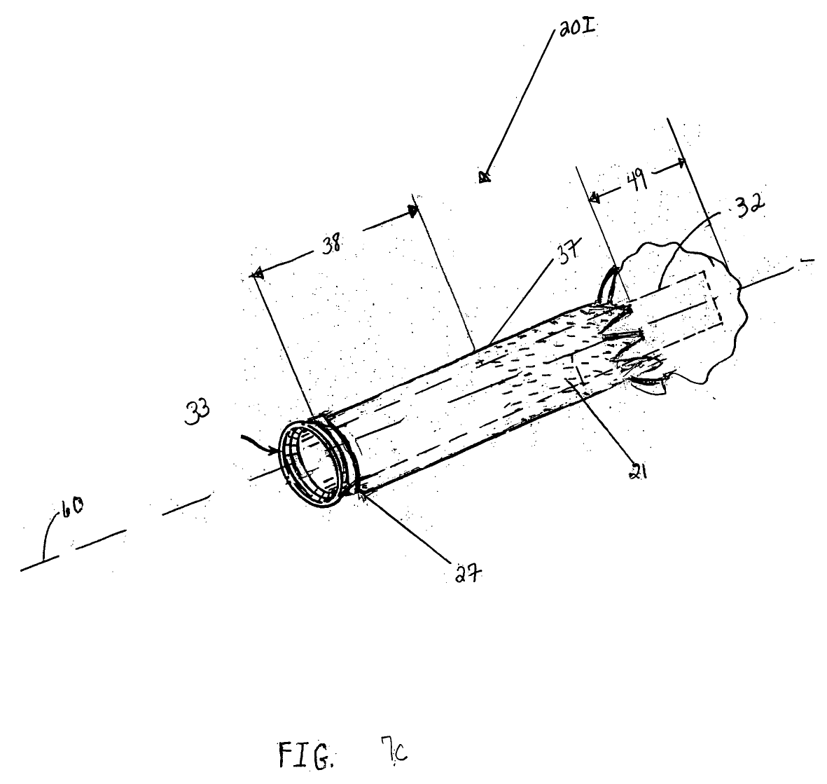

Below is a discussion of how the interaction of the tampon 21 and the inner member 24, 32 determines how the effective length 38 of the inner member 24 is measured. The tampon 21 could interact with the inner member in three ways. First, at least a portion of the tampon 21 could be embedded within the inner member 24 (See FIG. 3a). Second, the trailing end 37 of the tampon 21 could abut the first end 28 of the inner member 24 (See FIG. 6a). Third, the inner member 24 could be embedded in the tampon 21 (See FIG. 7a).

a. Tampon Embedded Within Inner Member

FIG. 3a is a perspective view of the inner member 24 separated from the hollow interior portion 33 of the outer member 23. Also, as seen in FIG. 3a, the tampon 21 can be embedded in at least a portion of the inner member 24 during the expulsion and/or before the expulsion of the tampon 21 from the tampon applicator 20A. The length that the tampon 21 is embedded within the inner member 24 is the embedded tampon length 48.

Also, as shown in FIG. 3a, tampon applicator 20A could have a tampon pushing member 51. The tampon pushing member 51 is located in the hollow interior portion 30 of the inner member 24. The tampon pushing member 51 positions the tampon 21 and provides the necessary force to the tampon 21 such that during expulsion of the exposed portion 49 (FIG. 4, FIG. 5) of tampon 21, the tampon 21 will travel a predetermined distance. The tampon pushing member 51 can be located in the inner member 24. The tampon pushing member 51 can be joined to the inner member 24. In one non-limiting example, as shown in FIG. 3a, the tampon pushing member 51 can be located adjacent to the first end 28 of the inner member 24 and the tampon pushing member 51 can be integral with the inner member 24. The tampon pushing member 51 in this limited embodiment can be created by four indented walls 31. The walls 31 can be indented along a portion of the length of the inner member 24. The portion of the inner member 24 which is indented creates a tampon pushing member 51 in which the trailing end 37 of the tampon 21 abuts the tampon pushing member 51 during expulsion of the exposed portion 49 (FIG. 4, FIG. 5) of tampon 21. In other words, an embedded portion 48 is contained within inner member 24 during expulsion of tampon 21.

FIG. 3b is a cross-section along lines 3b-3b of FIG. 3a. FIG. 3b shows tampon 21 adjacent to the tampon pushing member 51. The tampon pushing member 51 can be integral with the inner member 24. In this embodiment, tampon 21 is adjacent to the tampon pushing member 51.

FIG. 4 and FIG. 5 show alternative embodiments of the tampon applicator of the present invention when tampon 21 is embedded into inner member 24, 32 and when the tampon applicator is fully engaged resulting in the expulsion of the exposed portion 49 of the tampon 21. The tampon applicator of the present invention can be fully engaged when the second end 27 of outer member 23 becomes flush with the second end 29 of the inner member or after 1250 grams of force is applied to the inner member 32 along the longitudinal axis 60.

Referring to FIG. 4, when tampon 21 is embedded within the inner member 24 and when the inner member 24 becomes fully engaged by having second end 27 of the outer member 23 flush with the second end 29 of the inner member 24, the effective length 38 is the distance measured along the longitudinal axis 60 from the trailing end 37 of the tampon 21 to the second end 29 of the outer member 24. Alternatively, the effective length 38 can be calculated as the length of the inner member 24 minus the embedded tampon length 48. When tampon applicator 20B becomes fully engaged by having the second end 29 of inner member 24 becoming flush with second end 27 of the outer member 23, the exposed portion 49 of tampon 21 is expelled from tampon applicator 20B. In addition, when the second end 27 outer member 23 becomes flush with second end 29 of the inner member 24, the tampon 21 abuts the tampon pushing member 51 resulting in the exposed portion 49 expelling from tampon applicator 20B.

FIG. 5 shows tampon applicator 20C fully engaged after application of 1250 grams of force is applied to inner member 32 along the longitudinal axis 60. When the inner member 32 comes to a stop after applying 1250 grams of force to the inner member 32, the effective length 38 of the inner member 32 is the length as measured along the longitudinal axis 60 from the trailing end 37 of tampon 21 to the second end 27 of the outer member 23. In this embodiment, the inner member 32 comprises a hollow tube having a rim 33 which projects outwardly from the inner member 32. In addition, FIG. 5 shows an exposed portion 49 of tampon 21 after 1250 grams of force is applied to the inner member 32. In the embodiment of FIG. 5, after application of 1250 grams of force is applied to the second end 29 of inner member 32, the rim 33 of the inner member 32 prevents the inner member 32 from entering the outer member 23. In this embodiment, the rim 33 acts as a stop. A stop can be any feature that causes the inner member 32 to stop after 1250 grams of force is applied to the inner member 32.

b. Tampon Contacting Inner Member

FIG. 6a shows another alternative embodiment tampon applicator 20D. The first end 28 of the inner member 24 abuts the trailing end 37 of the tampon 21 during expulsion of the exposed portion 49 (see FIGS. 6b,6c) of the tampon 21. The second end 29 is the portion of the inner member 24 in which the axial force 44 is applied along the longitudinal axis 60 to expel the exposed portion 49 (see FIGS. 6b, 6c) of tampon 21 through the insertion end 26 of the outer member 23. The length of the inner member 24 is the effective length 38 in this embodiment.

FIG. 6b and FIG. 6c show alternative embodiments of the tampon applicator of the present invention when tampon 21 is not embedded into the inner member 24 and when the tampon applicator is fully engaged resulting in the expulsion of the exposed portion 49 of the tampon 21. The tampon applicator can be fully engaged when the second end 27 of the outer member 23 becomes flush with the second end 29 of the inner member 24 or after 1250 grams of force is applied to the inner member 32 along the longitudinal axis 60.

Referring to FIG. 6b, when the second end 27 of the outer member 23 becomes flush with the second end 29 of the inner member 24, the trailing end 37 of the tampon 21 abuts the first end 28 of the inner member 24 pushing the tampon 21 toward the insertion end 26 of the outer member 23 resulting in the exposed portion 49 expelling from tampon applicator 20B. In this embodiment, the first end 28 of the inner member 24 becomes the tampon pushing member 51. The length of the inner member 24 along the longitudinal axis 60 is the effective length 38 in this embodiment.

Referring to FIG. 6c, when the tampon 21 is not embedded into the inner member 32 and when the inner member 32 comes to a stop after applying 1250 grams of force to the inner member 32, the effective length 38 of the inner member 32 is the length measured along the longitudinal axis 60 from the trailing end 37 of the tampon 21 to the second end 27 of the outer member 23. In the embodiment of FIG. 6c, the point of stopping is the rim 33 which is the location which prevents the second end 29 of the inner member 32 from being inserted into outer member 23 after application of 1250 grams of force. The point of stopping may be different for different applicators. In this example, there can be a stop on the inner member 32 or the outer member 23. A stop can be any feature that causes the inner member 24 to stop after 1250 grams of force is applied. One skilled in the art could imagine that the inner member or the outer member 23 could have projections, nibs, ribs, nubs, etc. that can function to stop the inner member 24.

c. Inner Member Embedded Within the Tampon

FIG. 7a is a perspective view of the inner member 24 separated from the hollow interior portion 33 of the outer member 23. Moreover, FIG. 7a shows inner member 24 embedded within tampon 21. The length that the inner member 24 is embedded within tampon 21 is the embedded inner member length 50. In this embodiment, the first end 28 becomes the tampon pushing member 51. The trailing end 37 of tampon 21 defines the location of the first end 28 of the inner member 24. The effective length 38 of the inner member 24 is the length from the trailing end 37 of tampon 21 to the second end 29 of the outer member 23. When the inner member 32 comes to a stop after applying 1250 grams of force to the inner member 24, the effective length of the inner member 32 is defined by the trailing end 37 of tampon 21 to the second end 27 of the outer member 23.

FIG. 7b and FIG. 7c show alternative embodiments of the tampon applicator of the present invention when inner member 24, 32 is embedded into the tampon 21 and when the tampon applicator 20H, 201 is fully engaged resulting in the expulsion of the exposed portion 49 of the tampon 21. The tampon applicator of the present invention can be fully engaged when the outer member 23 becomes flush with the inner member 24 or after 1250 grams of force is applied to the inner member 32 along the longitudinal axis 60. FIG. 7b shows tampon applicator 20H becoming fully engaged when the second end 29 of outer member 23 becomes flush with the second end 27 of inner member 24 resulting in the exposed portion 49 expelling from tampon applicator 20H. The effective length 38 of the inner member 24 is measured along the longitudinal axis 60 from the trailing end 37 of the tampon 21 which abuts the first end 28 of inner member 24 to the second end 29 of the inner member 24. In this embodiment, the first end 28 becomes the tampon pushing member 51.

FIG. 7c shows tampon applicator 201 fully engaged after application of 1250 grams of force is applied to inner member 32 along the longitudinal axis 60. The inner member 32 can comprise a hollow tube having a rim 33 which projects outwardly from the inner member 32. In addition, FIG. 7c shows an exposed portion 49 of tampon 21 after 1250 grams of force is applied to the inner member 32. In this embodiment, the effective length 38 of the inner member 32 is measured from the trailing end 37 of the tampon 21 which abuts the inner member 32 to the point when the inner member 32 stops being inserted into the outer member 24. In this embodiment, the rim 33 is the stop.

In addition to delivering menstrual tampons into the vaginal cavity, it should be noted that the tampon applicator of the present invention can be used to deliver any other type of absorbent or nonabsorbent object to any suitable cavity. For example, the tampon applicator of the present invention could be used to insert incontinence inserts. An “incontinence insert,” as used herein refers to devices specifically designed, configured, and/or adapted for placement into a vagina in order to reduce the occurrence and/or severity of female urinary incontinence. While incontinence inserts are typically made of non-absorbent materials, at least partially absorbent materials may also be used. However, because there is no intent to absorb bodily fluids, and because the incontinence inserts are adapted and configured to provide structural support to the musculature and body tissues located near the urethra-vaginal myfascial area, incontinence inserts are readily distinguishable from catamenial tampons.

Below will provide more detail of each component of the tampon applicator of the present invention.

i. Outer Member

Referring to FIG. 2, typically, the outer member 23 can be used to handle or grip the tampon applicator during the insertion into the vaginal cavity. The outer member 23 is external to the inner member. The outer member 23 has a hollow interior 33, an insertion end 26, and a second end 27 opposed to the insertion end 26. The insertion end 26 is a portion of the outer member 23 in which the tampon applicator is inserted into the vaginal cavity and it is the end from which the tampon 21 is expelled. The second end 27 is opposed to the insertion end 26. The second end 27 can be the portion of the outer member 23 in which the user can handle or grip the tampon applicator. At least a portion of the hollow interior 33 of the outer member 23 engages with at least a portion of the inner member. In one non-limiting example, the inner member can be slideable within the outer member 23.

The manufacturer of the tampon applicator 20 can vary the size of the outer member 23. The size of the outer member 23 can be determined primarily by the dimensions of the tampon 21. Specifically, the diameter of the outer member 23 can be varied to accommodate different absorbency tampons. Generally, the outer member 23 can include an inner diameter from about 6 millimeters to about 16 millimeters and a wall thickness of from about 0.4 millimeter to about 0.6 millimeter. Generally, the inner diameter of the outer member 23 should be suitably greater than the diameter of the tampon 21 to prevent the outer member 23 from interfering with the removal of the tampon 21 from the outer member 23. Moreover, the inner diameter of the outer member 23 can have varying diameters and shapes to conform to the profiled shape of the enclosed tampon 21.

Also, the manufacturer of the tampon applicator can vary the length of the outer member 23. Generally, the outer member 23 should be of a sufficient length to house at least a portion of the tampon 21 prior to the expulsion of the tampon 21 from the tampon applicator into the vaginal cavity. Generally, the length of the outer member 23 can be any length. In one non-limiting example, the length of the outer member 23 can be less than 25 mm. In another non-limiting example, the length of the outer member 23 can be less than 35 mm. In yet another non-limiting example, the length of the outer member 23 can be less than 45 mm. In yet another non-limiting example, the outer member 23 can be from about 50 millimeters to about 85 millimeters. In yet another non-limiting example, the length of the outer member 23 is preferably from about 50 mm to about 100 mm, more preferably from about 55 mm to about 85 mm.

In addition, the manufacturer of the tampon applicator can vary the shape of the outer member 23. It is further noted herein that the shape of the outer member 23 can vary as long as a portion of the inner member can be slideable within the outer member 23. Moreover, the outer member 23 is in no way limited by the shape that it can assume except that the shape should not hinder directional expulsion of the tampon 21. One skilled in the art can imagine that the outer member 23 might be cylindrical or curved like a banana or any other suitable shape as long as any such shape would work effectively to allow the exposed portion 49 (FIG. 4, FIG. 5) of tampon 21 to expel from the tampon applicator and comfortably insert the tampon 21 into the vaginal cavity. The outer member 23 can be of any suitable cross-sectional shape. For example, suitable cross-sectional shapes can include, but are not limited to, circular, oval, flattened circular, elliptical, and any combination thereof.

In addition, the second end 27 can also have many designs. For example, the second end 27 can be scalloped at the second end 27 and/or have a feathered thickness edge.

Furthermore, the outer member 23 can contain the grip region 43 located adjacent to the second end 27 as noted above. The grip region 43 can provide for secure handling of the outer member 23. The perimeter of the grip region 43 can take essentially any desired shape, including oval, circular, and various other geometric forms. The gripping region 43 can be defined by outward projections or raised surfaces, created by impressing or compressing the surfaces. As seen in FIG. 2, the grip region 43 can be substantially circularly shaped, but it can also take on more angular formations such as squared. The surface of the grip region 43 can be any kind of surface known in the art. This surface can provide a desired frictional resistance for the fingers during the insertion of the tampon applicator 20 into the body. Moreover, this surface area can have, for example, as seen in FIG. 1, a flower pattern created via surface texturing, impressions, and/or indentations.

Furthermore, it is preferable that the grip region 43 be of sufficient dimension to substantially provide a comfortable grip for the user. As used in the specification, the term “user's grip” means any way of holding the tampon applicator in a hand, e.g., between a thumb and a finger. The grip region 43 need not necessarily extend completely around the perimeter of the outer member 23. For example, a space can be provided for a decorative marking or a Trademark character.

ii. Inner Member

Generally, the inner member 24 has a hollow interior 30, a first end 28, and a second end 29 opposed to the first end 28. The second end 29 is opposed to the first end 28. Moreover, the second end 29 is the portion of the inner member 24 in which the axial force 44 is applied to expel the tampon 21 from the outer member 23.

The inner member can have many different sizes and shapes. One skilled in the art can imagine many shapes of the inner member 24, for example, cylindrical or curved like a banana or any other suitable shape possible. The inner member can be of any suitable cross-sectional shape including, but are not limited to, circular, oval, flattened circular, elliptical, and any combinations thereof. For example, referring to FIG. 3a, the inner member 24 can be a hollow member having four indented walls 31. FIG. 3a shows tampon 21 adjacent to the tampon pushing member 51. The inner member 24 is integral with the tampon pushing member 51.

In another example, as seen in FIG. 5, the inner member 32 can comprise a hollow tube having a rim 33 which projects outwardly from the inner member 32. In addition, FIG. 5 shows an exposed portion 49 of tampon 21 after 1250 grams of force is applied to the inner member 32.

In FIG. 3a, the inner member can be designed to store at least a portion of the tampon 21 within its hollow interior 30 either during the expulsion and/or before the expulsion of the tampon 21 from the insertion end 26 of the outer member 23. Notably, the inner member can be shaped such that at least a portion of the tampon 21 is housed in or is partially contained by the inner member before the expulsion or during the expulsion of the tampon 21 from the tampon applicator 20.

The tampon pushing member 51 can be joined to the inner member 24 in any way known in the art. Specifically, the tampon pushing member 51 can be joined to the hollow interior 30 of the inner member by any known means in the art. Alternatively, the tampon pushing member 51 can be integral with the inner member.

The manufacturer of the tampon applicator of the present invention can vary the shape of the inner member as long as any such shape would work effectively to contain the embedded portion 48 of the tampon 21.

The size of the inner member can be determined primarily by the dimensions of tampon 21. Specifically, the diameter of the inner member can be varied to accommodate different absorbency tampons. For example, higher absorbency tampons can have larger diameters resulting in the inner diameter of the inner member having a larger diameter to house the higher absorbent tampon. In addition, the inner diameter of the inner member can have varying diameters and shapes to conform to the profiled shape of the enclosed tampon 21.

The length of the inner member can also vary. Generally, the inner member can be of a sufficient length to house a substantial portion of the tampon 21 prior to the insertion of the applicator into the body, properly aiding in the expulsion of the tampon 21 from the tampon applicator 20, and comfortably inserting into the vaginal cavity. For example, the length of the inner member 24 can be from about 43 millimeters to about 90 millimeters.

iii. Tampon Pushing Member

Referring to FIG. 3a, the tampon pushing member 51 positions the tampon 21 along the length of the inner member 24 so that during expulsion the exposed portion 49 of the tampon 21, the tampon 21 will travel a pre-determined distance.

The tampon pushing member 51 can be fixedly joined to the inner member in any way known in the art. The tampon pushing member 51 can also be monolithic with the inner member, for example, by molding the tampon pushing member 51 and the inner member as one piece. In one non-limiting example, the tampon pushing member 51 can be fixedly joined to the inner member by friction fitting that snaps parts together, gluing, and/or melting. In another non-limiting example, the tampon pushing member 51 can also be a continuation of the inner member by folding or compressing a portion of the inner member 24 and turning the inner member inwards upon itself to create a tube inside a tube.

FIG. 3b is a cross-section along lines 3b-3b of FIG. 3a. FIG. 3b shows tampon 21 adjacent to the tampon pushing member 51. The tampon pushing member 51 is integral with the inner member 24. In this embodiment, the tampon 21 is adjacent to the tampon pushing member 51.

Referring to FIG. 3a during the expulsion, at least a portion of the tampon 21 is adjacent to the tampon pushing member 51. In other words, when the tampon applicator 20A is fully engaged, the tampon pushing member 51 is in contact with the tampon 21. However, the tampon 21 may or may not be in contact with the tampon pushing member 51 before the expulsion of the exposed portion 49 (FIG. 4, FIG. 5) of tampon 21 from the tampon applicator 20.

It is further noted herein that the shape of the tampon pushing member 51 can vary as long as the tampon pushing member 51 aids in positioning the tampon 21 along the length of the outer member 23 to provide proper placement of the tampon 21 within the vaginal cavity. In one embodiment, the tampon pushing member 51 includes an elongated tampon positioning member of a generally tubular shape wherein the first end of the elongated tampon positioning member abuts the tampon 21 during expulsion. Alternatively, the tampon positioning member can be a rod wherein the first end of the rod abuts the tampon 21 during expulsion. In another embodiment, the tampon pushing member 51 can be slideable within the outer member 23. In another alternative embodiment, the tampon pushing member can also comprise projections extending inward from the hollow interior 33 of the inner member 24. The projections can be of any shape or size as long as the projections allow the tampon 21 to be in contact with the projections during the expulsion. The projections can be fixedly joined to the outer member 23 in any way known in the art.

It is further noted herein that the length of the tampon pushing member 51 can vary as long as the tampon pushing member 51 aids the tampon 21 to be located in a position within the inner member 24 that will provide low placement within the vaginal cavity. In one non-limiting example, the length of the tampon pushing member 51 can be from about 38.1 millimeters to about 57.15 millimeters.

In a preferred embodiment, as shown in FIG. 3a, the tampon pushing member 51 is monolithic with the inner member 24, for example, by molding the tampon pushing member 51. In this embodiment, the tampon positioning member is created in the form of indented walls 31 on a portion of the inner member 24. The portion which is indented creates a seat for the tampon 21 to rest.

C. Tampon Applicator Materials

Different tampon applicator parts can be constructed from different materials and processes. The tampon applicator or any part of the tampon applicator can be formed of a spirally wound, convolutedly wound, or longitudinally seamed hollow tube that is formed from paper, paperboard, cardboard, or any combinations thereof. The tampon applicator or any part of the tampon applicator can also be injection molded, extruded, or formed from flexible plastic, such as thermoformed from plastic sheet or folded or wound from plastic film.

The tampon applicator or any part of the tampon applicator can be constructed from a single ply of material or be formed from two or more plies that are bonded together to form a laminate. The use of two or more plies or layers is preferred for it enables the manufacturer to use certain materials in the various layers that can enhance the performance of the tampon applicator or any part of the tampon applicator. When two or more plies are utilized, all the plies can be spirally wound, convolutedly wound, or longitudinally seamed to form an elongated cylinder. The tampon applicator or any part of the tampon applicator can be constructed using a smooth thin ply of material on the outside or exterior surface that surrounds a coarser and possibly thicker ply. When the tampon applicator or any part of the tampon applicator contains at least three plies, the middle ply can be the thicker ply, and the interior and exterior plies can be smooth and/or slippery to facilitate the expulsion of the tampon and to facilitate the insertion of the tampon applicator or any part of the tampon applicator into a woman's vagina. By sandwiching a thick, coarser ply of material between two thin, smooth plies, a tampon applicator or any part of the tampon applicator can be provided which is very functional. The tampon applicator or any part of the tampon applicator should contain one to four plies, although more plies can be utilized if desired.

The plies forming the tampon applicator or any part of the tampon applicator can be held together by an adhesive, such as glue, heat, pressure, ultrasonic, or any combinations thereof. The adhesive can be either water-soluble or water-insoluble. A water-soluble adhesive is preferred for environmental reasons in that the tampon applicator or any part of the tampon applicator will quickly break apart when it is immersed in water. Such immersion will occur should the tampon applicator or any part of the tampon applicator be disposed of by flushing it down a toilet. Exposure of the tampon applicator or any part of the tampon applicator to a municipal's waste treatment plant wherein soaking in water, interaction with chemicals, and agitation all occur, will cause the tampon applicator or any part of the tampon applicator to break apart and evenly disperse in a relatively short period of time.

### EXAMPLES

The following is a listing of examples illustrating various embodiments of the present invention. It would be obvious to those skilled in the art that various other changes and modifications can be made without departing from the spirit and scope of the invention.

### Example 1

The tampon applicator of the present invention can comprise the outer member of the length of 45 millimeters, the inner member of the length of 70 millimeters, a tampon of the length of 44 millimeters, and 13 millimeters of the tampon embedded within the inner member.

### Example 2

The tampon applicator of the present invention can comprise the outer member of the length of 25 millimeters, the inner member of the length of 50 millimeters, a tampon of the length of 35 millimeters, and 17 millimeters of the tampon embedded within the inner member.

### Example 3

The tampon applicator of the present invention can comprise the outer member of the length of 35 millimeters, the inner member of the length of 55 millimeters, a tampon of the length of 40 millimeters, and 15 millimeters of the tampon embedded within the inner member.

### Example 4

The tampon applicator of the present invention can comprise the outer member of the length of 40 millimeters, the inner member of the length of 52 millimeters, a tampon of the length of 48 millimeters, and 12 millimeters of the tampon embedded within the inner member.

### Example 5

The tampon applicator of the present invention can comprise the outer member of the length of 25 millimeters, the inner member of the length of 70 millimeters, a tampon of the length of 48 millimeters, and 30 millimeters of the inner member embedded within the tampon.

### Example 6

The tampon applicator of the present invention can comprise the outer member of the length of 35 millimeters, the inner member of the length of 40 millimeters, a tampon of the length of 42 millimeters, and 18 millimeters of the inner member embedded within the tampon.

### Example 7

The tampon applicator of the present invention can comprise the outer member of the length of 45 millimeters, the inner member of the length of 73 millimeters, a tampon of the length of 46 millimeters, a tampon positioning member of the length of 20 millimeters, and 16 millimeters of the inner member embedded within the tampon.

### Example 8

The tampon applicator of the present invention can comprise the outer member of the length of 45 millimeters, the inner member of the length of 73 millimeters, a tampon of the length of 46 millimeters, a tampon positioning member of the length of 57 millimeters, and 16 millimeters of the inner member embedded within the tampon.

All documents cited in the Detailed Description of the Invention are incorporated, in relevant part, herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A tampon applicator for positioning a tampon inside a vaginal cavity, said applicator comprising:
a.) an outer member;
b.) an inner member slidable within said outer member, wherein said inner member has an effective length of less than about 58 mm; and
c.) at least a portion of said tampon being housed within said tampon applicator.

2. The tampon applicator according to claim 1 wherein said tampon is housed within said inner member.

3. The tampon applicator according to claim 1 wherein said tampon is housed within said outer member.

4. The tampon applicator according to claim 1 wherein said inner member has an effective length of less than about 56 mm.

5. The tampon applicator according to claim 1 wherein said inner member has an effective length of less than about 53 mm.

6. The tampon applicator according to claim 1 wherein said inner member has an effective length of less than about 45 mm.

7. The tampon applicator according to claim 1 wherein said inner member has an effective length of less than about 35 mm.

8. The tampon applicator according to claim 1 wherein said inner member has an effective length of less than about 25 mm.

9. The tampon applicator according to claim 1 wherein said outer member has a length of less than about 45 mm.

10. The tampon applicator according to claim 1 wherein said outer member has a length of less than 35 mm.

11. The tampon applicator according to claim 1 wherein said outer member has a length of less than 25 mm.
